(19) United States
(12) Patent Application Publication
(10) Pub. No.: US 2005/0283843 A1
(43) Pub. Date: Dec. 22, 2005

(54) Novel nuclear transfer technique through the processes of enucleation after activation to produce cloned mammals

(75) Inventors: Perng-Chih Shen, Tainan (TW); Feng-Hsiang Chu, Tainan (TW); Shan-Nan Lee, Tainan (TW); Winston Teng-Kuei Cheng, Taipei (TW)

Correspondence Address: NIKOLAI & MERSEREAU, P.A., 900 SECOND AVENUE SOUTH, SUITE 820, MINNEAPOLIS, MN, 55402, US

(73) Assignee: Livestock Research Institute, Council of Agriculture, Tainan (TW)

(21) Appl. No.: 10/869,427

(22) Filed: Jun. 16, 2004

(51) Int. Cl. A01K 67/027

(52) U.S. Cl. 800/14; 800/21; 800/15

(57) ABSTRACT

The present invention relates to a method for producing a mammal reconstruction cell, a method for producing a nuclear transferred (NT) embryo capable of developing into a non-human mammal, a method for producing a non-human mammal fetus, a method for producing a non-human mammal and the products prepared by the foregoing methods.

### BACKGROUND OF THE INVENTION

1. Field of the Invention

The present invention relates to a method for producing a mammal reconstruction cell, a method for producing a nuclear transferred (NT) embryo capable of developing into a non-human mammal, a method for producing a non-human mammal fetus, a method for producing a non-human mammal and the products prepared by the foregoing methods.

2. Description of Related Art

Cloning animals by using nuclear transfer of somatic cells has become possible. The enucleation before activation (EBA) method is a conventional method of producing an embryo and comprises enucleating oocytes, transferring donor cells into oocytes, fusing the oocytes and the donor cells and activating the fused reconstruction cells. However, low cloning efficiency due to the mortality of the embryo at early stage, gestation period and post partum, organ hypoplasia, large offspring syndrome, etc, have all been experienced with EBA (Campbell et al., 1996, Wilmut et al., 1997, Schnieke et al., 1997, Renard et al., 1999 and Wells et al., 1999). These abnormal conditions may be caused by unable to reprogram the transferred donor cells for the development of the embryos (Kang et al., 2001 and Xue et al., 2002).

The reprogramming of mammalian genes is generally related to gene imprinting. If a somatic gene expressed in tissue-specific or developmental stage-specific manner, the somatic gene is usually an imprinted gene (Barlow, 1995).

Mammals are capable of transmitting the imprinted gene phenomena to progenies. When an imprinted gene in a chromosome is developing to primordial germ cells (PGC) in gametogenesis, a large part of the methylation phenomenon will be erased (Monk et al. 1987 and Labosky et al., 1994). When gametes are maturing, the implanted gene in female and male gametes will be reestablished to the correct methylation model. The chromosome is highly methylated in matured oocytes and sperms, indicating the methylated genes in oocytes belong to maternal imprinted genes, and the methylated genes in sperms belong to paternal imprinted genes (Falls et al., 1999 and Davis et al., 2000). When the sperms and oocytes are fertilized, genomic DNA will be genome-wide demethylated until the fertilized oocytes develop to blastocyst stage (Monk et al., 1987, Howlett and Reik, 1991 and Kafri et al., 1992) and then continuously establish de novo methylation until finishing gene imprinting (Howell et al., 1998 and Hsieh, 2000).

Low cloning efficiency is resulted from the nucleus of NT embryos could not proceed in the proper epigenetic reprogramming as normal embryos. The abnormal phenomena were existed in cloned embryos during the preimplantation stage embryo (Kang et al., 2001) and new born cloned animals (Xue et al., 2002). Furthermore, gene reprogramming is closely linked with DNA methylation. The objective was to improve the cloning efficiency by the method of novel nuclear transfer procedures for decreasing the methylation of the NT embryos.

### SUMMARY OF THE INVENTION

An aspect of the present invention is to provide a method for producing a mammal reconstruction cell comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid cell;
(e) activating the tetraploid cell; and
(f) enucleating the oocyte in the activated tetraploid cell to generate a diploid reconstruction cell.

Another aspect of the present invention is to provide a mammal reconstruction cell prepared by the foregoing method.

Yet another aspect of the present invention is to provide a method for producing a nuclear transfer (NT) embryo capable of developing into a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo; and
(g) culturing the diploid NT embryo.

Still another aspect of the present invention is to provide a nuclear transfer (NT) embryo capable of developing into a non-human mammal prepared by the foregoing method.

Still another aspect of the present invention is to provide a method for producing a non-human mammal fetus, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus.

Still another aspect of the present invention is to provide a non-human mammal fetus prepared by the foregoing method.

Still another aspect of the present invention is to provide a method for producing a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to produce a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus that undergoes full fetal development and parturition to generate a live-born mammal.

Still another aspect of the present invention is to provide a non-human mammal prepared by the foregoing method.

Further benefits and advantages of the present invention will become apparent after a careful reading of the detailed description.

### DETAILED DESCRIPTION OF THE INVENTION

A preferred method for producing a nuclear transfer embryo in accordance with the present invention is fusing oocytes and donor cells to generate reconstruction cells and activating the reconstruction cells to generate tetraploid nuclear transfer embryos and then enucleating the nuclear of the oocytes to generate normal diploid embryos. The nuclear of the oocytes' transient existence help the donor cells demethylation.

Mammals undergo genome-wide demethylation twice during development. The first genome-wide demethylation occurs in PGC cells at the stage of developing gametes. The aim of the first genome-wide demethylation is to transmit the imprinting gene phenomena to progenies. The second genome-wide demethylation occurs after oocyte and sperm fertilization to improve the zygote restored developmental totipotency (Monk et al., 1987 and Labosky et al., 1994). The cause for the genome-wide demethylation of the zygote was earlier considered that no factor for maintaining methylation exists after embryo cleavage. The replication of chromosome is increased and the methylation rate is progressively decreased (Howlett and Reik, 1991). However, studies in recent years have shown DNA demethylase is another key factor for this phenomenon and the main function of the DNA demethylase is actuating demethylation (Cervoni et al., 1999). A recent study discovered that the timing for demethylation of female and male pronuclear to occur in zygote is different. Demethylation of the male pronuclear occurred several hours after the gametes have been fertilized, but the female pronuclear was progressively decreased following the progress of embryo cleavage. The phenomenon suggests that demethylation of male pronuclear is induced actively by the demethylated factor existing in oocyte. The demethylation of the female pronuclear is passively and progressively finished following DNA replication in embryo cleavage (Haaf, 2001).

The term “nuclear transfer embryo” as used herein refers to an embryo that is produced by transferring a donor cell or the nuclear thereof into an oocyte.

The term “serum starvation” as used herein refers to cells that culture in a medium containing a low amount of fetal calf serum.

The term “transgenic cell” as used herein refers to a cell that an exogenous DNA sequence has been added by recombinant DNA technology.

A preferred embodiment of the present invention is to provide a method for producing a mammal reconstruction cell comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid cell;
(e) activating the tetraploid cell; and
(f) enucleating the oocyte in the activated tetraploid cell to generate a diploid reconstruction cell.

A preferred embodiment of the invention is to provide a method for producing a nuclear transferred (NT) embryo capable of developing into a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo; and
(g) culturing the diploid NT embryo.

A preferred embodiment of the present invention is to provide a method for producing a non-human mammal fetus, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus.

A preferred embodiment of the present invention is to provide a method for producing a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus that undergoes full fetal development and parturition to generate a live-born mammal.

In a preferred embodiment of the present invention, the mammal preferably is livestock and more preferably, the mammal is bovine.

In a preferred embodiment of the present invention, the oocyte provided in step (a) is preferably matured in vivo or in vitro and contains the first polar body.

In a preferred embodiment of the present invention, the donor cell provided in step (b) preferably is a somatic cell and more preferably, the somatic cell is a transgenic cell.

In a preferred embodiment of the present invention, the donor cell provided in step (b) preferably is serum starved.

In a preferred embodiment of the present invention, the donor cell provided in step (b) preferably is not serum starved.

In a preferred embodiment of the present invention, step (c) is preferably transferring the donor cell into the perivitelline space of the oocyte.

In a preferred embodiment of the present invention, step (c) is directly injecting the donor cell into the cytoplasm of the oocyte.

In a preferred embodiment of the invention, the oocyte in step (d) is not enucleated.

In a preferred embodiment of the invention, the fusion of step (d) is using an electrical stimulus.

In a preferred embodiment of the invention, the activation of step (e) is incubating the fused cell in an activating solution comprising calcium ionophore (A23187) and 6-dimethylaminopurine (6-DMAP). The conventional EBA method comprises enucleating oocytes, transferring donor cells into oocytes, fusing the oocytes and the donor cells and activating the fused NT embryos. A preferred method of producing an embryo in accordance with the present invention is enucleation after the activation (EAA) method, which comprises transferring donor cells into oocytes, fusing the oocytes and the donor cells, activating the fused NT embryos and enucleating the oocytes. In the following example, the EAA method and EBA method are used for producing NT-embryos present closely in vitro development rate in different stages and this suggests that using the EAA method for producing NT-embryos will not damage the viability of in vitro development.

Two of the recipient females were successfully pregnant and gave birth to two calves after transferring of 20 NT-embryos into 11 recipient females of which produced with EBA method. In contrast, one of the recipient females was pregnant and one calf was born after transferring of 2 NT-embryos into a recipient female of which produced by EAA method. The result showed that the NT-embryos prepared by the two methods were capable of developing to individuals. Further study of the methylation rates of the donor cells and the NT-embryos prepared by the EBA method showed that the methylation rates of the donor cells and the NT-embryos prepared by the EBA method were close (69.78% & 64.66%). However, these results were significantly higher than the rates of the embryos produced from in vitro production (IVP) (31.87%) and the NT-embryos produced by the EAA method (44.42%)(p<0.001). The rate of the NT-embryos produced by the EAA method was higher than that of the IVP (p<0.001). The results suggest that the methylation rate from development to blastocyst was decreased in normal embryos produced from in vitro culture. The NT-embryos produced by the EBA method maintained the original higher methylation status as to the donor cells. However, the methylation rate of the NT-embryos produced by the EAA method decreased.

Since the tetraploid embryos could not be developed to full term, the present invention is performing enucleation of oocyte nuclei after finishing the fusion and activation process of NT-embryos to generate the diploid NT-embryos.

The in vitro development capability of the NT-embryos produced by the EAA and EBA method are very similar. However, the methylation rate of the NT-embryos produced by the EAA method is lower than that by the EBA method. The lower methylation rate of NT-embryos is closest to the condition of in vitro produced embryos. Furthermore, the donor cells used in the present invention may be transgenic cells. The donor cells may be inserted the gene that can translate functional proteins e.g. blood clotting factors in the chromosome of the donor cells. The inserted gene may clone in an appropriate location of the chromosome for specific expression in milk to increase the utility of cloned animals.

The new nuclear transfer method in accordance with the present invention can be applied to clone animals and also can set up a perfect technical platform for studying cloned animals. The genetic products produced by the transgenic cloned animals may be particularly competitive in international markets.

All of the documents or publications recited in the text are incorporated herein by reference.

Further details of this invention are illustrated in the following examples.

### EXAMPLE 1

Preparation of Oocyte

1.1. Materials

The ovary used in this example was taken from the Taiwan Yellow Cattle and the Holstein from the slaughterhouse. The physiological background of the cattle was not clear.

1.2. Process

After slaughtering of the cattle, the ovaries of the cattle were excided and immersed in physiological saline containing 0.1 mg/ml of penicillin-streptomycin (Gibco, 15140-122) at about 30° C. The ovaries were washed in the foregoing physiological saline, spread with 70% ethanol and this process was repeated two times. The ovaries were put in a cleaned 10 cm dish and the contents that included oocyte-cumulus complex (COC) and follicular fluid in follicles (2-8 mm diameter) on the surfaces of the ovaries were sucked by a 1 ml syringe with No. 18 needle under negative pressure. The sucked contents were put on a low powered (20-30×) 3-D dissecting microscope and the total COCs that were suspended in the follicular fluid were collected by pasteur pipette with an appropriate caliber and the quality well and cumulus cells completed oocytes were chosen. The oocytes were washed with a pre-warmed (37° C.) maturation medium 3-5 times.

### EXAMPLE 2

Maturation of Oocyte

2.1. Preparation of the Maturation Medium

9.5 ml of medium-199 (Gibco, 12340-030) were added with 0.5 ml of fetal bovine serum (FBS, Gibco, 10270-106) and 1 μl solution containing 5 mg/ml gentamycin to prepare the maturation medium. The maturation medium was filtered and packaged to refrigeration (4° C.).

2.2. Maturation of the Oocyte

According to the bovine oocyte maturation step described by Lee et al (1997), 4 drops each containing 100 μl of maturation medium were prepared, covered with an overlay of mineral oil (sigma, M-8410) in a 35 mm culture dish, incubated at 38.5° C. in 2% CO₂, 95% air and saturated humidity and incubated for at least 4 hours incubation before culturing. Ten to twenty of the COCs prepared in example 1 were transferred in the foregoing maturation medium and cultured in the same previous condition for 18-19 hours. The in vitro matured oocytes containing the first polar body were chosen.

### EXAMPLE 3

In Vitro Production of Bovine Embryos

3.1. Preparation of Sperm

3.1.1. Preparation of BO Basic Solution

According to the described method of Brackett and Oliphant (1975), 6.55 g of NaCl, 0.3 g of KCl, 0.115 g of NaH₂PO₄H₂O, 0.106 g of MgCl₂.6H₂O and 0.331 g CaCl₂.2H₂O were dissolved in the sterile distilled water and quantified to 100 ml for producing the BO basic solution. The prepared BO basic solution was filtered and packaged for frozen (−20° C.).

3.1.2. BO Operative Solution

0.3104 g of NaHCO₃ and 0.0138 g of Na-pyruvate were dissolved in 70 ml of BO basic solution, quantified to 100 ml by adding sterile distilled water and adjusting the pH to 7.6-7.8 for producing BO operative solution. The prepared BO operative solution was filtered and packaged for refrigeration (4° C.).

3.1.3. Sperm Washing Solution

0.3604 g of theophylline (Sigma, T-1633) were dissolved in 200 ml of BO operative solution to prepare 10 mM theophylline of sperm washing solution. The sperm washing solution was refrigerated (4° C.).

3.1.4. Sperm Capacitation Solution

0.05 g of heparin (Sigma, H-3149) were dissolved in 10 ml of sterile distilled water to prepare 5 mg/ml heparin stock solution. The heparin stock solution was filtered, packaged and frozen (−20° C.). 100 μl of the heparin stock solution and 0.5 g bovine serum albumin (Fraction V, BSA; Sigma, A-6793) were dissolved in 50 ml BO operative solution to prepare 10 μg/ml heparin, 10 mg/ml BSA sperm capacitation solution. The sperm capacitation solution was filtered, packaged and frozen (−20° C.).

3.2. Process for Sperm Capacitation

According to the method described by Parrish et al. (1986), the frozen semen of the Holstein breed was taken from liquid nitrogen and directly put in 37° C. warm water for 30 seconds. One ml of thawed semen and 10 ml of sperm washing solution were mixed well, centrifuged (800 g) for 5 min, then the supernatant was removed, and the foregoing steps were repeated once. After the sperms were washed two times, 1 ml of the retained semen was added to 1 ml of sperm capacitation solution (the concentration of the sperm was about 1×10⁷ cells/ml). After mixing well, 0.5 ml of semen was put in a 35 mm culture dish and covered with an overlay of mineral oil to prepare sperm suspending drops. The sperm suspending drops were incubated in constant temperature incubator for 15 min to carry out capacitation.

3.3. Process for In Vitro Fertilization

After the bovine oocyte prepared in example 2 was in vitro maturated over 24 hours, the cumulus cells around the oocytes were slightly removed by gentle pipetting. The oocytes were then put in the capacitated sperm suspending drops and transferred to the constant temperature incubator for external fertilization. After 8 hours, the oocytes were sucked by the pasteur pipette, put in the maturation medium, the sperm around them was removed by mechanically continuously sucking and splitting, and then washed with a maturation medium three times. The final oocytes were put back in the maturation medium with monolayer cumulus cells for co-culture (Lee et al. 1997).

### EXAMPLE 4

Preparation of Donor Cell

4.1. Primary Culture of Bovine Ear Cell

The method for culturing the ear cell line of the adult Holstein Cattle was according to the method described by Kubota et al. (1998). The desired ear tissue was shaved and washed and then the desired ear tissue was sterilized with 70% ethanol. The desired ear tissue was cut using sterile scissors into several sections each about 0.5×1 cm² and then put into 0.9% normal saline with 0.1 mg/ml penicillin/streptomycin. The ear tissue was washed with the foregoing conditional normal saline three times, put into 70% ethanol for 5 min, washed with the foregoing conditional normal saline three times, put in a 1.5 ml centrifugal tube, cut using sterile scissors into about 3 mm³, transferred to 6 cm culture dish and then cultured with 10% FCS DMEM (Gibco, 11965-092) at 37° C. in 5% CO₂ and saturated humidity condition for 10-14 days. The supernatant of the culture dish was removed, and 3 ml of solution containing 0.25% trypsin were immediately added to suspend the cells. 6 ml of the culture medium were added to stop the activation of the trypsin and the supernatant was collected. The collected supernatant was centrifuged (250 g) at room temperature for 5 min, the supernatant was removed, 1 ml cell culture medium added to disperse the pallet, the dispersed pallet was transferred to a 10 cm culture dish, 9 ml of cell culture medium added to the culture dish and mixed well and then cultured at 37° C. in 5% CO₂, 95% air and saturated humidity condition. During the culture period, the cell culture medium was changed 2-3 times each week to produce fresh cell culture medium. After the cell growth filled the culture dish (about 2×10⁷ cells), the foregoing culture step was repeated to carry out secondary culture. After the ear cells passed through secondary culture twice, the supernatant was collected and centrifuged and then 4 ml of freezing preservative solution containing 10% DMSO (dimethylsulfoxide, Sigma, D-5879) and 90% fetal calf serum were added and mixed well. The concentration of the cells was adjusted to 5×10⁶ cells/ml. The cells were then packaged in a freezing tube (1 ml/tube) and put in turn at −20° C. for 4 hours, −80° C. for 16-18 hours and preserved in liquid nitrogen (−196° C.) for long term preservation. If the cells were to directly play roles of donor cells after thawing, the concentration of the cells was adjusted to 5×10⁵ cells/ml and packaged to freeze.

4.2. Starvation of the Donor Cell

The method for starvation of the donor cell was modified by the method described by Wilmut et al (1997). The frozen cells in the tube prepared as foregoing were taken from liquid nitrogen, immediately thawed in a 37° C. water bath, 3 ml of culture medium DMEM containing 10% FCS were added to the tube, transferred to a 35 mm culture dish for 4 hours and then changed to the fresh culture medium. After the cells growth filled the culture dish, the cells were suspended and transferred to 4-well plate for culture. After the cells were grown to about 80% of full size, the cell culture medium was changed to DMEM containing 0.5% FCS for starvation over 5-8 days. One part of the starved cells was used as donor cells. Another part of the starved cells was counted to about 10⁰⁰ cells as one unit, put in 0.5 ml of centrifugal tube, centrifuged and the supernatant was removed, 2 μl of mPBS solution added for rapidly freezing in liquid nitrogen and preserved at −80° C. refrigerator for analysis of the methylation of the DNA.

### EXAMPLE 5

Production of Nuclear Transfer (NT) Embryo

5.1. Enucleation of the Bovine Oocyte

5.1.1. Preparation of Hoechst 33342 Fluorescence Agent

The method for preparation of Hoechst 33342 fluorescence agent was modified by the method described by Mohamed et al. (1999). 10 mg of Hoechst 33342 (Sigma, B-2261) were dissolved in 10 ml of sterile water for preparation of fluorescence stock solution at 1 mg/ml Hoechst 33342 and packaged to be frozen at −20° C. Before use, 2 μl of the fluorescence stock solution and 198 μl of M-199 culture medium containing 5% FCS were mixed well to dilute as Hoechst 33342 operative solution at 10 μg/ml.

5.1.2. Confirmation of the Enucleation Rate of the Bovine Oocytes with Hoechst 33342 Staining

A. Conventional Method of Enucleation before Activation (EBA)

According to Kubota et al. (1998), the matured COCs were removed from the external cumulus cells and the oocytes with PBI were put into a micromanipulating chamber. Enucleation was achieved by cutting the zona pellucida then removing the PBI and its surroundings with a glass needle. The enucleated oocytes, the PBI and the cytoplasma were individually put in 5% FCS M-199 culture drops. The PBI and the cytoplasma were then stained by 110 μg/ml Hoechst 33342 for 20 min. The successful rate of the enucleation was analyzed by UV light at 343 nm wave length. If the cytoplasma compressed out from the oocytes were stained with blue fluorescence, this showed that the nuclear was removed.

B. Invented Method of Enucleation after Activation (EAA)

The donor cell was transferred into the unenucleated oocyte and the reconstruction cells were fused to generate tetraploid nuclear transfer embryo (NT-embryo). After activating of the tetraploid NT-embryo, the NT-embryo was enucleated to restore the chromosome as diploid. The confirmation of the enucleation rate of the bovine oocytes was the same as the conventional method. If the cytoplasma compressed out from the oocytes were stained with one blue fluorescence point, this showed that the nuclear was removed.

5.2. Micromanipulation for Putting the Donor Cells into the Perivitelline Space of the Oocytes

The enucleated oocytes or the unenucleated oocytes and the donor cells were individually put into injection drops. The field of the microscope was firstly moved to the injection room containing donor cells and the pipette sucked an appropriate amount (about 10 cells each time) of donor cells. The field of the microscope was moved to the injection room containing oocytes to transfer the donor cells into the oocytes. The step was to firstly hold the oocytes by use of an holding pipette and the pipette was operated to contain donor cells for insertion into the cut that was formed in enucleated step, whereby one donor cell was put into the perivitelline space of the oocyte. In the unenucleated oocyte group, the donor cells were put in the location perpendicular to the PBI to prevent the donor cells from removing in the enucleation step.

### EXAMPLE 6

Fusion of the NT-embryo

6.1. Preparation of the Fusion Solution

6.1.1. Preparation of the CaCl₂ Stock Solution

0.011 g of CaCl₂ were dissolved in 10 ml of sterile distilled water to form 10 mM CaCl₂ stock solution, packaged and refrigerated at 4° C.

6.1.2. Preparation of the MgSO₄ Stock Solution

0.023 g of MgSO₄ were dissolved in 10 ml of sterile distilled water to form 10 mM MgSO₄ stock solution, packaged and refrigerated at 4° C.

6.1.3. Preparation of the Fusion Solution

2.915 g of mannitol (Sigma, M-9546) were dissolved in 40 ml of sterile distilled water and added to 500 μl of CaCl₂ stock solution, 500 μl MgSO₄ stock solution and 0.5 mg of BSA to be mixed well and quantified to 50 ml by sterile distilled water to form 0.32 M mannitol+100 μM CaCl₂+100 μM MgSO₄+0.01 mg/ml BSA fusion solution. The fusion solution was packaged and refrigerated at 4° C. Before use, the fusion solution was pre-warmed to room temperature (25-30° C.). Furthermore, the fusion solution was mixed with different ratios of embryo culture medium to prepare 4 drops each 80 μl individually containing 25%, 50%, 75% and 100% of fusion solution in a 35 mm culture dish and then overlaid with mineral oil. The culture dish was incubated for at least 4 hours.

6.2. Electrical Fusion

The method for fusion of the oocytes and the donor cells was modified by the method described by Kubota et al. (1998). Before electrical fusion, the NT-embryo was transferred in a drop containing 25% fusion solution and in turn transferred to a drop containing 50%, 75% and 100% fusion solution each for 2 min.

1.5 ml of the fusion solution was put in a 100 mm embryo culture dish to form an electrical fusion chamber. Two sterile microneedles were mounted in the chamber to adjust the direction of the NT-embryo and make the contact surfaces between the donor cells and the oocytes parallel with the tangent planes of the end of the microneedles and make the direction of the electrical field perpendicular to the contact surfaces. An electrocell manipulator (ECM 2001, BTX Inc., San Diego, USA) was actuated to fuse the donor cells and the oocytes with a fusion pulse (15 μsec) and direct current at 1.5 kV/cm. The fused NT-embryo was transferred to a drop containing a 50% fusion solution for 2 min and then transferred to an embryo culture medium and cultured in constant temperature incubator. After 30 min, the fusion rate of NT-embryo was observed with a handstand microscope.

### EXAMPLE 7

Activation of the NT-Embryo

7.1. Preparation of the Activation Solution

7.1.1. Preparation of the Calcium Ionophore (A23187) Activation Solution

1 mg of calcium ionophore (Sigma, C-7522) was dissolved in 1.91 ml of DMSO to prepare 1 mM stock solution, packaged and frozen at −20° C. Before use, the stock solution was thawed at room temperature and 5 μl of the stock solution were added to 995 μl of an embryo culture medium and mixed well to prepare 5 μM working solution.

7.1.2. Preparation of the 6-Dimethylaminopurine (6-DMAP) Activation solution

100 mg of 6-DMAP (Sigma, D-2629) were added in 3.064 ml of TCM-199 culture medium and put in water bath at 56° C. to prepare 200 mM stock solution and packaged to be frozen at −20° C. Before use, the stock solution was thawed at room temperature and 10 μl of the stock solution were added to 990 μl of an embryo culture medium and mixed well to prepare 2 mM working solution. 4 drops each 50 μl were put in a 35 mm culture dish and overlaid with mineral oil and incubated in the embryo incubator for at least 4 hours.

7.2. Process for Activation

The method for activation was modified by the method described by Liu et al. (1998). Four hours after electric fusion, the foregoing fused EBA and EAA NT-embryos were incubated within 5 μM A23187 medium for 5 min and then put in the activation solution containing 2 mM 6-DMAP for 4 hours.

### EXAMPLE 8

Culture of the NT-Embryo and the Fertilized Embryo

8.1. Preparation of Culture Drop

The method for preparation of culture drop containing monolayer cumulus cells was modified by the method described by Lee et al. (1997). When the foregoing COCs cultured for 18-19 hours were removed from the cumulus cells, the remaining cumulus cells in the original matured culture drop were continuously cultured to grow and fill the culture drop such that the external culture drop with monolayer cumulus cells was formed.

8.2. In Vitro Culture of Embryo

The fertilized embryo prepared in example 3 and the activated NT-embryo prepared in example 7 were washed with embryo culture medium three times, and then put into the prepared culture drop with monolayer cumulus cells and incubated in a constant temperature incubator. The development of the embryo was observed over a period of 24 hours after incubation had started. The culture drop was changed every two days to fresh culture drop until the 8^{th} day after incubation had started. After the fertilize embryos and the NT-embryos developed to blastocysts, a part of the NT-embryos were transferred to recipient animals. Another part of the NT-embryos and the in vitro production (IVP) embryos were washed with mPBS three times, loaded into a 0.5 ml centrifuge tube for centrifugation. Afterwards, the tube was directly put into the liquid nitrogen and immediately into −80° C. refrigerator for preservation and DNA methylation analysis. During the procedure of collecting the genomic DNA, it is necessary to thaw the embryos with 37° C. warm water and freeze with liquid nitrogen repeatedly in order to destroy the zone pellucida.

### EXAMPLE 9

Transfer the NT-Embryo into a Recipient

The blastocyst stage NT-embryos produced by the EBA and EAA methods were transferred by a non-surgical method into the uterus of a recipient where the estrous cycle of the cow was synchronized with the development stage of the embryo. Before the NT-embryos were transferred into the uterus of the cows, the NT-embryos were transferred into an M-199 medium containing 20% FCS. Methods for transferring embryos into the uterus of the cow are well known in the art and thus further description thereof is omitted.

### EXAMPLE 10

### Effect of NT Methods on the In Vitro Development and Pregnancy of the NT-Embryo

10.1. Process

The foregoing bovine oocytes cultured in vitro for 18-19 hours were removed from the cumulus cells and divided into two groups. One group of the oocytes was prepared to produce the NT-embryos by the EBA method and the other was prepared by the EAA method. The NT-embryos produced by the two methods were transferred into the embryo culture drop prepared in example 8 at 38.5° C. in 2% CO₂ and in a saturated humidity incubator for 8 days. The development of the NT-embryos was observed over a period of 24 hours after culture had started. When the NT-embryos had developed to be blastocyst, the NT-embryos were transferred by non-surgical method into the synchronized cows. After 53 days of implantation, the pregnancy of the recipient was confirmed by rectum palpation for determination of the in vivo development of NT-embryos.

10.2. Result

The effect of nuclear transfer methods on the in vitro development of NT-embryos cloned with bovine ear fibroblast cells is shown in Table 1.

TABLE 1

| | No. of | | | | |
|---|---|---|---|---|---|
| NT | cultured NT- | No. (%) of NT-embryos developed to^{★} | | | |
| method* | embryos | 2-cell | 16-cell | CM | B |
| EBA | 82 | 76(92.7) | 48(58.5) | 41(50.0) | 38(46.3) |
| EAA | 85 | 75(88.2) | 49(57.6) | 41(48.2) | 38(44.7) |

*EBA: Enucleation before activation; EAA: Enucleation after activation.
^{★}CM: Compacted molura; B: Blastocyst.

The results show that percentages of the in vitro development of the NT-embryos produced by the two methods were close to each other. The two groups had no significant difference (p>0.05).

The pregnancy of NT-embryos produced by different nuclear transfer methods is shown in Table 2.

TABLE 2

| | No. of NT- | No. of | |
|---|---|---|---|
| NT | embryos | recipient | No. (%) of |
| method* | transferred | females | pregnancy |
| EBA | 20 | 11 | 2 |
| EAA | 2 | 1 | 1 |

After 20 NT-embryos produced by EBA method was transferred into 11 recipient bovines, 2 recipients were pregnant and produced 2 healthy cloned calves. Two NT-embryos produced by EAA method were transferred into one recipient, the recipient cow was pregnant and gave birth to a healthy cloned calf

### EXAMPLE 11

Test of the DNA Methylation of the NT-Embryo

The method for analyzing DNA methylation was modified by the method described by Warnecke et al. (1998) and Kang et al. (2001).

11.1. Chemical Treatment of Bisulfite
(1) The rapid frozen and thawed IVP embryos and the NT-embryos prepared in example 8 and the donor cells prepared in example 4 were individually added to 2 μl of 1 μg/μl of E. coli tRNA, 2 μl of 0.02 M of SDS, 0.3 μl of 19 g/μl of proteinase K and 14.7 μl of distilled water to a final volume 20 μl at 37° C. in a water bath for 1 hour;
(2) Reacting at 98° C. for 15 min;
(3) Adding 2.5 μl BamHI and 2.5 μl of 10× buffer at 37° C. for 15 min;
(4) Adding 2 μl of 3M NaOH at 37° C. for 15 min;
(5)Adding 12 μl of 10 mM hydroquinone, 208 μl of 2.3M sodium metabisulfite (pH=5) and 2 μl of 1 μg/μl of E. coli tRNA at 50° C. for 10 hours;
(6) Using DNA purifying kit (Wizad DNA clean up system, Promega, A-7280) to purify DNA, remove salts in the DNA sample and obtain 40 μl of DNA solution;
(7) Adding 4.4 μl of 3M NaOH at 37° C. for 15 min;
(8) Adding 2 μl of 1 μg/μl of E. coli tRNA, 28 μl of 5 M ammonium acetate (pH=7) and 180 μl of 100% ethanol and mixing well to precipitate DNA;
(9) Centrifuging (10000 G) for 10 min and removing the supernatant; and
(10) Drying DNA and dissolving in 20 μl of distilled water and freezing at −20° C.

11.2. Amplification of Bisulfite Treated DNA by Polymerase Chain Reaction (PCR)

The amplified fragment for analysis DNA methylation was located in bovine satellite I gene. The sequence of the sense primer BSI (+) and the antisense primer BSI(−) were shown in Kang et al., Nat. Genet. 28: 173-177. 2001.

The BSI (+) primer was marked with FAM fluorescence. The method comprises
(1) Taking 4 μl of the foregoing bisulfite treated DNA solution;
(2)Adding 0.75 μl of 10 pmol/μl BSI(+) and BSI (−) primers, 1 μl of 5 U/μl EX Taq, 4 μl of 50 μM dNTP, 2 μl of EX Taq specified 10× buffer and 11.5 μl sterile distilled water to a final volume 20 μl; and
(3) Putting in a PCR machine at (I) 94° C. over 60 sec for one cycle; (II) 94° C. over 60 sec, 46° C. over 60 sec and 72° C. over 20 sec for 40 cycles; (III) 72° C. over 5 min; and (IV) 4° C. for stop.

11.3. Analysis of DNA Methylation

The principle of analyzing DNA methylation was using bisulfite to convert the non-methylated cytosine in the DNA sequence to uracil and the methylated cytosine was maintained. The treated DNA was cut by restriction enzyme that cut specified the DNA methylation location. The maintained cytosine was cut by the restriction enzyme and the converted uracil was not cut by the restriction enzyme (Warnecke et al. 1998).

The full length of the foregoing amplified satellite I gene is 211 bp. The fragment has 12 methylation locations (CpG1-CpG12) and the methylation locations and the converted sequence treated bisulfite with were shown in Kang et al. (2001).

In the reference, each CpG is numbered and framed. Two AciI recognition sequences (5′-CCGC-3′) encompass CpG4 and CpG7. If this DNA fragment was in methylated status, the Acil will cut CpG4 and CpG7 to 35, 86 and 90 bp fragments. Since only BSI (+) was marked, the detected fragments were 90 bp, 125 bp and 211 bp fragments. The process involved taking 4 μl of PCR products, adding 2 μl of AciI restriction enzyme (NEB, ROSSIS), 2 μl Acil specified 10× buffer and 12 μl of sterile distilled water to final volume 20 μl and reacting at 37° C. for 12 hours. 1 μl of the products was added to 0.5 μl of TAMRA fluorescence marked 350 bp molecular size standard (Applied Biosystems, 401736), 0.5 μl buffer and 3 μl of distilled formamide solution and mixed well, denatured at 90° C. for 2 min and immediately put on ice. 1.5 μl of the denatured solution was loaded in the gel for electrophoresis of an autosequence machine (Applied Biosystems-377) for over 2 hours and the amount of the fragments in the gel were analyzed using GeneScan and Genotyper software (Applied Biosystems, Foster City, USA). The total amount of the fragments (including about 90 and 125 bp fragments) that were cut by Acil, divided by the amount of DNA sample (including all cut or non-cut DNA) were the methylation ratio of satellite I gene.

11.4. Result

The methylation rates of satellite I gene in IVP-embryos, donor cells, and NT-embryos which were produced by EBA and EAA methods are shown in Table 3.

TABLE 3

| | No. of | Methylation rates of satellite I gene (%) |
|---|---|---|
| Treatment* | replicates | Mean (SD) |
| IVP | 6 | 31.87 (±4.83)^{c} |
| Donor cell | 6 | 69.78 (±5.62)^{a} |
| EBA-NT | 6 | 64.66 (±1.66)^{a} |
| EAA-NT | 6 | 44.42 (±2.96)^{b} |

*IVP: Embryos produced from in vitro culture; EBA: NT-embryos produced by enucleation before activation method; EAA: NT-embryos produced by enucleation after activation method.
^{a,b,c}Values without the same superscripts in the same columns are significantly different (p < 0.001).

The results show that the methylation rates of the donor cells and the NT-embryos produced by the EBA method were close (69.78% & 64.66%). However, both were significantly higher than the rates of the IVP (31.87%) and the EAA method (44.42%)(p<0.001). The rate of the NT-embryos produced by the EAA method was significantly higher than that of the IVP (p<0.001).

### EXAMPLE 12

Statistical Analysis

The statistical analysis for the in vitro development of the NT-embryos produced by the EBA and EAA methods used in example 10 was by Chi-square analysis. The methylation rates of the embryos were analyzed by General Linear Models to carry out analysis of variance and further confirmed by the Duncan mean comparative method to estimate the difference of the two methods.

Various modifications and variations of the present invention will be recognized by those persons who were skilled in the art without departing from the scope and spirit of the invention. Although the invention has been described in connection with specific preferred embodiments, the invention as claimed should not be unduly limited to such specific embodiments. Indeed, various modifications of the described modes for carrying out the invention, which are obvious to those skilled in the art, are intended to be within the scope of the following claims.

Reference

Barlow, D. P. 1995. Genomic imprinting in mammals. Science 270:1610-1613.
Brackett, B. G. and G. Oliphant. 1975. Capacitation of rabbit spermatozoa in vitro. Biol. Reprod. 12:260-274.
Campbell, K. H., J. McWhir, W. A. Ritchie and I. Wilmut. 1996. Sheep cloned by nuclear transfer from a cultured cell line. Nature 380:64-66.
Cervoni, N., Sanjoy Bhattacharya and Moshe Szyf. 1999. DNA demethylase is a processive enzyme. J. Biol. Chem. 274:8363-8366.
Davis, T. L., G. J. Yang, J. R. McCarrey and M. S. Bartolomei. 2000. The H19 methylation imprint is erased and re-established differentially on the parental alleles during male germ cell development. Hum. Mol. Genet. 9:2885-2894.
Falls, J. G., D. J. Pulford, A. A. Wylie and R. L. Jirtle. 1999. Genomic imprinting: implications for human disease. Am. J. Pathol. 154:635-647.
Haaf, T. 2001. The battle of the sexes after fertilization: behaviour of paternal and maternal chromosomes in the early mammalian embryo. Chromosome Res. 9:263-271.
Howell, C. Y., A. L. Steptoe, M. W. Miler and J. R. Chaillet. 1998. Cis-acting signal for inheritance of imprinted DNA methylation patterns in the preimplantation mouse embryo. Mol. Cell. Biol. 18: 4149-4156.
Howlett, S. K. and W. Reik. 1991. Methylation levels of maternal and paternal genomes during preimplantation development. Development 113:119-127.
Hsieh, C. L. 2000. Dynamics of DNA methylation pattern. Cour. Opin. Genet. Dev. 10:224-228.
Kafri, T., M. Ariel, M. Brandeis, R. Shemer, L. Urven, J. McCarrey, H. Cedar and A. Razin. 1992; Developmental pattern of gene-specific DNA methylation in the mouse embryo and germ line. Genes Dev. 6:705-714.
Kang, Y. K., D. B. Koo, J. S. Park, Y. H. Choi, A. S. Chung, K. K. Lee and Y. M. Han. 2001. Aberrant methylation of donor genome in cloned bovine embryos. Nat. Genet. 28: 173-177.
Kubota, C., X. Yang, A. Dinnyes, J. Todoroki, H. Yamakuchi, K. Mizoshita, S. Inohae and N. Tabara. 1998. In vitro and in vivo survival of frozen-thawed bovine oocytes after IVF, nuclear transfer, and parthenogenetic activation. Mol. Reprod. Dev. 51:281-286.
Labosky, P. A., D. P. Barlow and B. L. M. Hogan. 1994. Mouse embryonic germ (EG) cell lines: transmission through the germline and defferences in the compared with embryonic stem (ES) cells. Development 120:3197-3204.
Lee S. N., C. F. Lieu and Y. M. Hsu. 1997. Development of bovine oocytes matured, fertilized and co-cultured with cumulus cells in vitro. J. Chin. Soc. Anim. Sci. 26(4):429-438.
Liu, L. C. Ju and X. Yang. 1998. Parthenogenetic development and protein patterns of newly matured bovine oocytes after chemical activation. Mol. Reprod. Dev. 49:298-307.
Mohamed Nour, M. S. and Y. Takahashi. 1999. Preparation of young preactivated oocytes with high enucleation efficiency for bovine nuclear transfer. Theriogenology 51:661-666.
Monk, M., M. Boubelik and S. Lehnert. 1987. Temporal and regional changes in DNA methylation in the embryonic, extraembryonic and germ cell lineages during mouse embryo development. Development 99:371-382.
Parrish, J. J., J. L. Susko-Parrish, M. L. Leibfried-Rutledge, E. S. Critser, W. H. Eyestone and N. L. First. 1986. Bovine in vitro fertilization with frozen-thawed semen. Theriogenology 25: 591-600.
Renard, J. P., S. Chastnat, P. Chesne, C. Richard, J. Marchal, N. Cordonnier, P. Chavatte and X. Vignon. 1999. Lymphoid hypoplasis and somatic cloning. Lancet 353:1489-1491.
Schnieke, A. E., A. J. Kind, W. A. Ritchie, K. Mycock, A. R. Scott, M. Ritchie, I. Wilmut, A. Colman and K. H. S. Campbell. 1997. Human factor IX transgenic sheep produced by transfer of nuclei from transfected fetal fibroblasts. Science 278: 2130-2133.
Warnecke, P. M., J. R. Mann, M. Frommer and S. J. Clark. 1998. Bisulfite sequencing in preimplantation embryos: DNA methylation profile of the upstream region of the mouse imprinted H19 gene. Genomics 51:182-190.
Wells, K. N., P. M. Misica and H. R. Tervit. 1999. Production of cloned calves following nuclear transfer with cultured adult mural granulose cells. Biol. Reprod. 60: 996-1005.
Wilmut, I., A. E. S., J. McWhir, A. J. Kind and K. H. S. Campbell. 1997. Viable offspring derived from fetal and adult mammalian cell. Nature 385: 810-813.
Xue, F., X. C. Tian, C. Kubota, F. Du, M. Taneja, A. Dinnyes, Y Dai, H. Levine, L. V. Pereira and X. Yang. 2002. Aberrant X-chromosome inactivation in deceased cattle derived from somatic cloning. Nat. Genet. 31: 216-220.

1. A method for producing a mammal reconstruction cell comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid cell;
(e) activating the tetraploid cell; and
(f) enucleating the oocyte in the activated tetraploid cell to generate a diploid reconstruction cell.

2. The method as claimed in claim 1, wherein the mammal is livestock.

3. The method as claimed in claim 2, wherein the mammal is bovine.

4. The method as claimed in claim 3, wherein the oocyte provided in step (a) is matured in vivo or in vitro and contains a first polar body.

5. The method as claimed in claim 3, wherein the donor cell provided in step (b) is a somatic cell.

6. The method as claimed in claim 5, wherein the somatic cell is a transgenic cell.

7. The method as claimed in claim 3, wherein the donor cell provided in step (b) is serum starved.

8. The method as claimed in claim 3, wherein the donor cell provided in step (b) is not serum starved.

9. The method as claimed in claim 3, wherein the step (c) is transferring the donor cell into the perivitelline space of the oocyte.

10. The method as claimed in claim 3, wherein the step (c) is directly injecting the donor cell into the cytoplasm of the oocyte.

11. The method as claimed in claim 3, wherein the oocyte in step (d) is unenucleated.

12. The method as claimed in claim 3, wherein the fusion of step (d) is using an electrical stimulus.

13. The method as claimed in claim 3, wherein the activation of step (e) is incubating the fused cell in an activating solution comprising calcium ionophore and 6-dimethylaminopurine (6-DMAP).

14. A mammal reconstruction cell produced by the method as claimed in claim 1.

15. A method for producing a nuclear transferred (NT) embryo capable of developing into a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo; and
(g) culturing the diploid NT embryo.

16. The method as claimed in claim 15, wherein the mammal is livestock.

17. The method as claimed in claim 16, wherein the mammal is bovine.

18. The method as claimed in claim 17, wherein the oocyte provided in step (a) is matured in vivo or in vitro and contains a first polar body.

19. The method as claimed in claim 17, wherein the donor cell provided in step (b) is a somatic cell.

20. The method as claimed in claim 19, wherein the somatic cell is a 7 transgenic cell.

21. The method as claimed in claim 17, wherein the donor cell is serum starved.

22. The method as claimed in claim 17, wherein the donor cell is not serum starved.

23. The method as claimed in claim 17, wherein the step (c) is transferring the donor cell into the perivitelline space of the oocyte.

24. The method as claimed in claim 17, wherein the step (c) is directly injecting the donor cell into the cytoplasm of the oocyte.

25. The method as claimed in claim 17, wherein the oocyte in step (d) is unenucleated.

26. The method as claimed in claim 17, wherein the fusion of step (d) is using an electrical stimulus.

27. The method as claimed in claim 17, wherein the activation of step (e) is incubating the fused tetraploid NT embryo in an activating solution comprising calcium ionophore and 6-dimethylaminopurine (6-DMAP).

28. A nuclear transferred (NT) embryo capable of developing into a non-human mammal produced by the method as claimed in claim 15.

29. A method for producing a non-human mammal fetus, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus.

30. The method as claimed in claim 29, wherein the mammal is livestock.

31. The method as claimed in claim 30, wherein the mammal is bovine.

32. The method as claimed in claim 31, wherein the oocyte provided in step (a) is matured in vivo or in vitro and contains a first polar body.

33. The method as claimed in claim 31, wherein the donor cell provided in step (b) is a somatic cell.

34. The method as claimed in claim 33, wherein the somatic cell is a transgenic cell.

35. The method as claimed in claim 31, wherein the donor cell is serum starved.

36. The method as claimed in claim 31, wherein the donor cell is not serum starved.

37. The method as claimed in claim 31, wherein the step (c) is transferring the donor cell into the perivitelline space of the oocyte.

38. The method as claimed in claim 31, wherein the step (c) is directly injecting the donor cell into the cytoplasm of the oocyte.

39. The method as claimed in claim 31, wherein the oocyte in step (d) is unenucleated.

40. The method as claimed in claim 31, wherein the fusion of step (d) is using an electrical stimulus.

41. The method as claimed in claim 31, wherein the activation of step (e) is incubating the fused tetraploid NT embryo in an activating solution comprising calcium ionophore and 6-dimethylaminopurine (6-DMAP).

42. A non-human mammal fetus prepared by the method as claimed in claim 29.

43. A method for producing a non-human mammal, the method comprising:
(a) providing a mammalian oocyte;
(b) providing a mammalian donor cell;
(c) transferring the donor cell or the nucleus thereof into the oocyte;
(d) fusing the donor cell or the nucleus thereof with the oocyte to generate a tetraploid NT embryo;
(e) activating the tetraploid NT embryo;
(f) enucleating the oocyte in the tetraploid NT embryo to generate a diploid NT embryo;
(g) culturing the diploid NT embryo; and
(h) transferring the cultured diploid NT embryo into a recipient mammal female so as to produce a mammal fetus that undergoes full fetal development and parturition to generate a live-born mammal.

44. The method as claimed in claim 43, wherein the mammal is livestock.

45. The method as claimed in claim 44, wherein the mammal is bovine.

46. The method as claimed in claim 45, wherein the oocyte provided in step (a) is matured in vivo or in vitro and contains a first polar body.

47. The method as claimed in claim 45, wherein the donor cell provided in step (b) is a somatic cell.

48. The method as claimed in claim 47, wherein the somatic cell is a transgenic cell.

49. The method as claimed in claim 45, wherein the donor cell is serum starved.

50. The method as claimed in claim 45, wherein the donor cell is not serum starved.

51. The method as claimed in claim 45, wherein the step (c) is transferring the donor cell into the perivitelline space of the oocyte.

52. The method as claimed in claim 45, wherein the step (c) is directly injecting the donor cell into the cytoplasm of the oocyte.

53. The method as claimed in claim 45, wherein the oocyte in step (d) is unenucleated.

54. The method as claimed in claim 45, wherein the fusion of step (d) is using an electrical stimulus.

55. The method as claimed in claim 45, wherein the activation of step (e) is incubating the fused tetraploid NT embryo in an activating solution comprising calcium ionophore and 6-dimethylaminopurine (6-DMAP).

56. A non-human mammal prepared by the method as claimed in claim 43.
